(19) United States
(12) Patent Application Publication
(10) Pub. No.: US 2025/0124815 A1
(43) Pub. Date: Apr. 17, 2025

(54) SYSTEMS AND METHODS FOR GENERATING CUSTOMIZED MEDICAL SIMULATIONS

(71) Applicant: INTUITIVE SURGICAL OPERATIONS, INC., Sunnyvale, CA (US)

(72) Inventors: Anthony M. Jarc, Johns Creek, GA (US); Joey Chau, Cupertino, CA (US); May Quo-Mei Liu, Rougemont, NC (US)

(21) Appl. No.: 18/847,050

(22) PCT Filed: Mar. 14, 2023
(86) PCT No.: PCT/US2023/064324

§ 371 Date: Sep. 13, 2024

Related U.S. Application Data
(60) Provisional application No. 63/320,553, filed on Mar. 16, 2022.

(51) Int. Cl. G09B 23/28; G09B 9/00; G16H 10/60; G16H 40/63; G16H 50/00

(52) U.S. Cl. CPC G09B 23/286; G09B 9/00; G16H 10/60; G16H 40/63; G16H 50/00

(57) ABSTRACT

A medical system may comprise a display system, an operator input device and a control system in communication with the display system and the operator input device. The control system may comprise a processor and a memory comprising machine readable instructions that, when executed by the processor, cause the control system to access an experience factor for a user and reference a set of parameterized prior procedures. The instructions may also cause the control system to identify a parameterized prior procedure associated with the experience factor from the set of parameterized prior procedures and generate a simulation exercise that includes a plurality of parameters from the parameterized prior procedure. Model inputs to the operator input device that are associated with the plurality of parameters may be determined.

### CROSS-REFERENCED APPLICATIONS

This application claims priority to and benefit of U.S. Provisional Application No. 63/320,553, filed Mar. 16, 2022 and entitled “Systems and Methods for Generating Customized Medical Simulations,” which is incorporated by reference herein in its entirety.

### FIELD

The present disclosure is directed to systems and methods for generating a customized medical simulation.

### BACKGROUND

Minimally invasive medical techniques are intended to reduce the amount of tissue that is damaged during diagnostic or surgical procedures, thereby reducing patient recovery time, discomfort, and harmful side effects. Such minimally invasive techniques may be performed through one or more surgical incisions or through natural orifices in a patient anatomy. Through these incisions or natural orifices, clinicians may insert minimally invasive medical instruments to conduct medical procedures by manually or by a robot-assisting actuation of the instrument. To improve medical procedures, train clinicians, and/or evaluate the effectiveness of medical procedures, customized medical simulations may be developed.

### SUMMARY

Examples of the invention are summarized by the claims that follow the description. Consistent with some examples, a medical system may comprise a display system, an operator input device and a control system in communication with the display system and the operator input device. The control system may comprise a processor and a memory comprising machine readable instructions that, when executed by the processor, cause the control system to access an experience factor for a user and reference a set of parameterized prior procedures. The instructions may also cause the control system to identify a parameterized prior procedure associated with the experience factor from the set of parameterized prior procedures and generate a simulation exercise that includes a plurality of parameters from the parameterized prior procedure. Model inputs to the operator input device that are associated with the plurality of parameters may be determined.

Consistent with some examples, a method for generating a customized medical simulation exercise may comprise accessing an experience factor for a user, referencing a set of parameterized prior procedures and identifying a parameterized prior procedure associated with the experience factor from the set of parameterized prior procedures. The method may also include generating a simulation exercise that includes a plurality of parameters from the parameterized prior procedure and determining model inputs to an operator input device that are associated with the plurality of parameters.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a method of customizing a simulation exercise, according to some embodiments.

FIG. 2 illustrates a user profile, according to some embodiments.

FIG. 3 is a schematic illustration of a parameterized medical procedure record, according to some embodiments.

FIG. 4 is a flowchart illustrating measurement and record systems for parameterizing a medical procedure.

FIG. 5A is a method of generating a customized simulation exercise, according to some embodiments.

FIG. 5B is a method of generating a customized simulation exercise, according to some embodiments.

FIG. 5C is a method of generating a customized simulation exercise, according to some embodiments.

FIG. 6 is a schematic view of a robot-assisted medical system according to some embodiments.

FIG. 7 is a perspective view of a manipulator assembly according to some embodiments.

FIG. 8 is a perspective view of an operator input system according to some embodiments.

Embodiments of the present disclosure and their advantages are best understood by referring to the detailed description that follows. It should be appreciated that like reference numerals are used to identify like elements illustrated in one or more of the figures for purposes of illustrating but not limiting embodiments of the present disclosure.

### DETAILED DESCRIPTION

A medical skill development system may identify a user's skill development need and may generate a customized simulation syllabus, including one or more simulation exercises, built from prior procedure data. The simulation exercises may strengthen the user's skill competencies. Systems and methods are provided for generating customized medical procedure simulations. Clinician profiles and parameterized prior procedures may be used to create simulation exercises that address the development needs of the clinician.

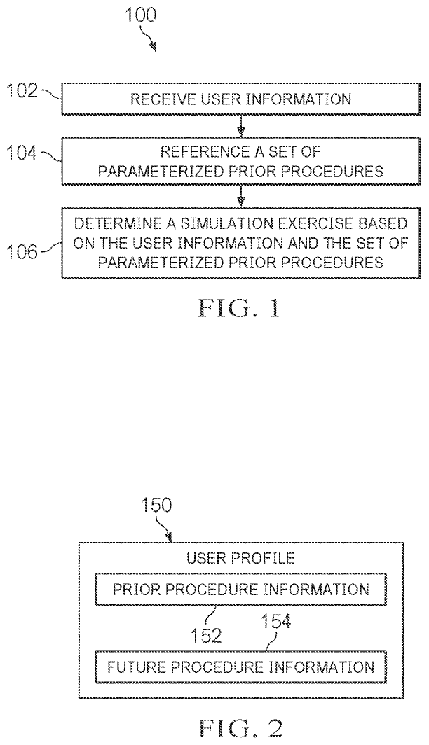

FIG. 1 illustrates a method 100 of producing a simulation exercise, according to some embodiments. The method 100 is illustrated as a set of operations or processes. The processes illustrated in FIG. 1 may be performed in a different order than the order shown in FIG. 1, and one or more of the illustrated processes might not be performed in some embodiments of method 100. Additionally, one or more processes that are not expressly illustrated in FIG. 1 may be included before, after, in between, or as part of the illustrated processes. In some embodiments, one or more of the processes of method 100 may be implemented, at least in part, in the form of executable code stored on non-transitory, tangible, machine-readable media that when run by one or more processors (e.g., the processors of a control system) may cause the one or more processors to perform one or more of the processes. At a process 102, user information may be received. At a process 104, a catalog, database, or set comprising one or more parameterized prior procedures may be referenced. At a process 106, a simulation exercise may be determined based on the user information and the referenced parameterized prior procedures.

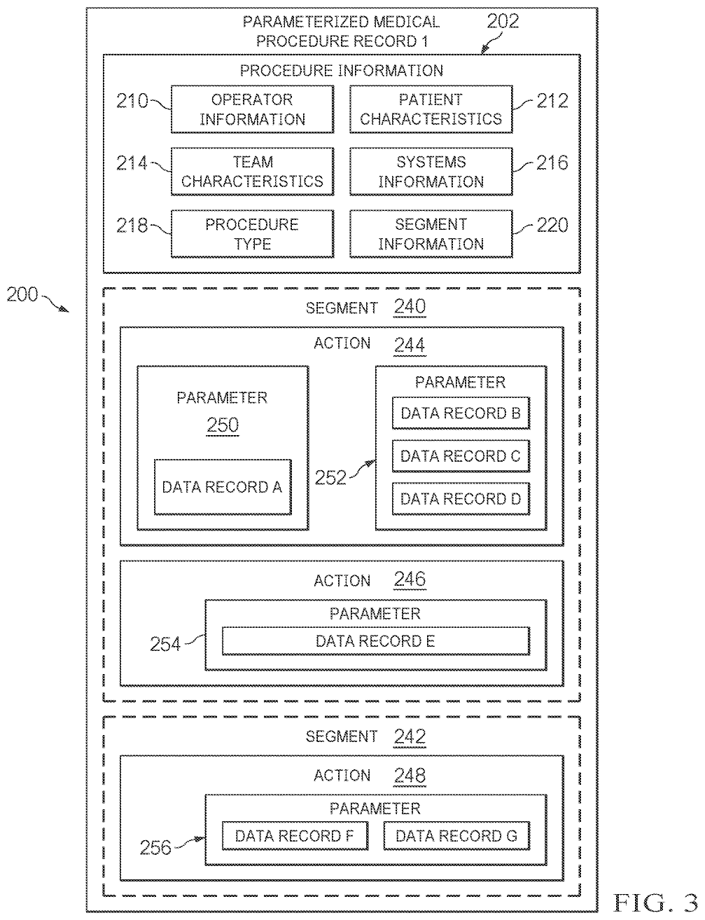

In greater detail, at the process 102, user information may be received from a user profile. A user experience factor may be determined from the user profile and may include a skill development subject for the user to improve a clinical skill or may include a type of future procedure scheduled for the user to allow the user to prepare for a future experience. For example, the user information may be received at a processor (e.g., processor 620 of medical system 610). In some examples, the medical procedure record may be received from a memory device (e.g., a memory 624 of medical system 610). FIG. 2 illustrates an example of a user profile 150 including prior procedure information 152 and future or prospective procedure information 154. The user may be, for example, a surgeon, medical device operator, surgical assistant, or other clinician or operator involved in a medical procedure. In some examples, the user may be a synthetic user with artificial intelligence that may be trained using the methods and systems described herein. Prior procedure information 152 may include, for example, information about patients involved in prior procedures performed by the user. Patient information or characteristics may include data from the patient electronic medical record, gender, height, weight, body mass index, medical image data (e.g., CT images, ultrasound images), medical measurement data (e.g., blood pressure, EKG results), and/or other information about patients involved in the user's prior procedures. Prior procedure information 152 may additionally or alternatively include, for example, team characteristics for prior medical procedures performed by the user Team characteristics may include, for example, the roles of team members involved in the prior procedures, the experience levels of each team member involved in the prior procedures, and the prior experience of each team member in collaboration with the user. Prior procedure information 152 may additionally or alternatively include, for example, information about surgical systems or instruments used in prior medical procedures performed by the user. The surgical systems or instruments may include, for example, robot-assisted medical systems, laparoscopic instruments, open procedure instrument, or any combination thereof. Prior procedure information 152 may additionally or alternatively include, for example, procedure type information for prior medical procedures performed by the user. The types of procedures may include, for example abdominal, cardiac, colorectal, gynecological, head/neck, pulmonary, thoracic, and/or urology procedures. Prior procedure information 152 may additionally or alternatively include, for example, segmental information about prior medical procedures performed by the user. Segmental information may include, for example, difficulty information about the prior procedure or segments of the prior procedure. Prior procedure information 152 may additionally or alternatively include, for example, a user skill profile including assessment information from prior medical procedures performed by the user. The assessment information may include performance evaluations based on objective performance indicators. The assessment information may include evaluations of technical aspects of the prior procedure, including information about errors, inefficiencies, suboptimal patient outcomes, or other positive or negative indicia of the user's technical proficiency. The assessment information may also or alternatively include evaluations of non-technical aspects of the prior procedure, including information about communication effectiveness, leadership, stress, or other positive or negative indicia of the user's non-technical proficiency. Prior procedure information may be generated, for example, from procedures performed on a patient, during a training session with synthetic tissue structures, or during a prior computer-generated simulation.

The future procedure information 154 may include information about scheduled, planned, or otherwise known or expected future procedures to be performed by the user. For each future procedure, various information may be received, including, for example, patient information, team composition information, surgical system information for the systems and devices to be used, the procedure type, procedure segment information, and/or the expected level of difficulty.

Referring again to FIG. 1, at the process 104, a parameterized prior procedure catalog, database, or other information set may be referenced or interrogated. During a medical procedure, sensor systems, measurement systems, and/or recording systems may capture and store data records in a medical procedure record. In some examples, the medical procedure record may capture information about a medical procedure performed with a robot-assisted medical system, with a laparoscopic medical system, with a manual medical device, or with a combination of systems and devices. Each medical procedure record may capture information about a medical procedure performed on a patient by a single clinician or a team of medical professionals. As described in U.S. Provisional Patent Application No. 63/320,538. entitled “Systems and Methods for Parameterizing Medical Procedures, filed Mar. 16, 2022, which is incorporated by reference herein in its entirety, medical procedures may be parameterized. Parameters may be associated with characteristic actions of medical procedures and may be based on data records generated during the prior medical procedures. A catalog, database or set of parameterized procedures may include parameterized procedures associated with a particular clinician; parameterized procedures from a guide, trainer, or other expert; parameterized procedures from other users (e.g. a peer, a trainee); and/or parameterized procedures from synthetic procedures performed virtually or on a synthetic patient.

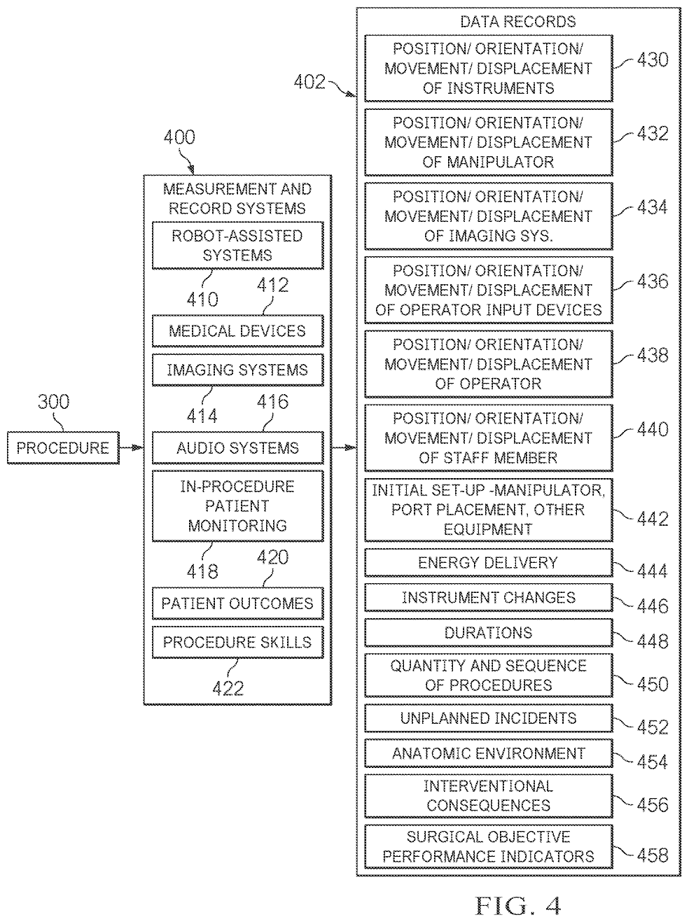

FIG. 3 is a schematic illustration of a parameterized medical procedure record 200 that may be stored, for example, in the set of parameterized prior procedures. The parameterized medical procedure record 200 may include procedure information 202 and the data records A-G from the medical procedure associated with the procedure information 202. The procedure information 202 may include a variety of input parameters to the procedure. For example, operator information 210 may include the clinician identification information, training history, experience, preferences, and/or other information related to one or more clinicians involved in the medical procedure. The operator may be, for example, a surgeon, a surgical trainee, an expert or guide medical practitioner. The procedure information 202 may include patient characteristics 212 including patient age, gender, height, weight, medical history and/or other information related to the unique patient on which the medical procedure was performed. The procedure information may include team characteristics 214 for the team members and support staff involved in the medical procedure including identification information, role in the procedure, training history, experience, preferences, or other information related to the personnel performing the procedure. The procedure information 202 may also include system information 216 about the system or systems used to perform the procedure. The systems may include sensors or recording systems that capture information such as settings or sensed parameters for the system during a procedure. Systems may include a robot-assisted medical system or components thereof, a laparoscopic medical system, a manual medical device, and/or support or peripheral systems. The system information 216 may include manufacturer, model, serial number, time in service, time since last maintenance, count of use cycles, maintenance history, calibration information, or other information related to systems used to perform the medical procedure. The procedure information 202 may also include procedure type 218 which includes information about the type of medical procedure. Medical procedure type may include abdominal, cardiac, colorectal, gynecological, neurological, head/neck, pulmonological, thoracic, urologic, or other category or subcategory of anatomic system involved in the medical procedure. The procedure information 202 may also include segment information 220. Segment information 220 may include information about subdivided portions of the medical procedure. For example, procedure segments may include sequences or groups of actions associated with ablation, stapling, suturing, dissection, tissue resection, anastomosis, camera control, instrument wrist control, setting changes, or tool changes that occur once or multiple times during a medical procedure. In some examples, procedure information may also include other information about the procedure including, for example, the date on which the procedure occurred, the time and duration of the procedure, and/or the location and facility identification where the procedure occurred.

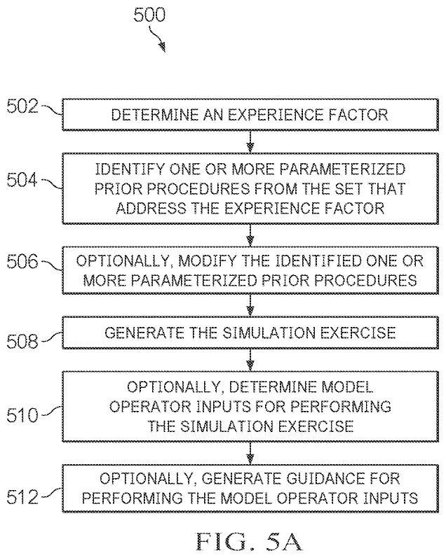

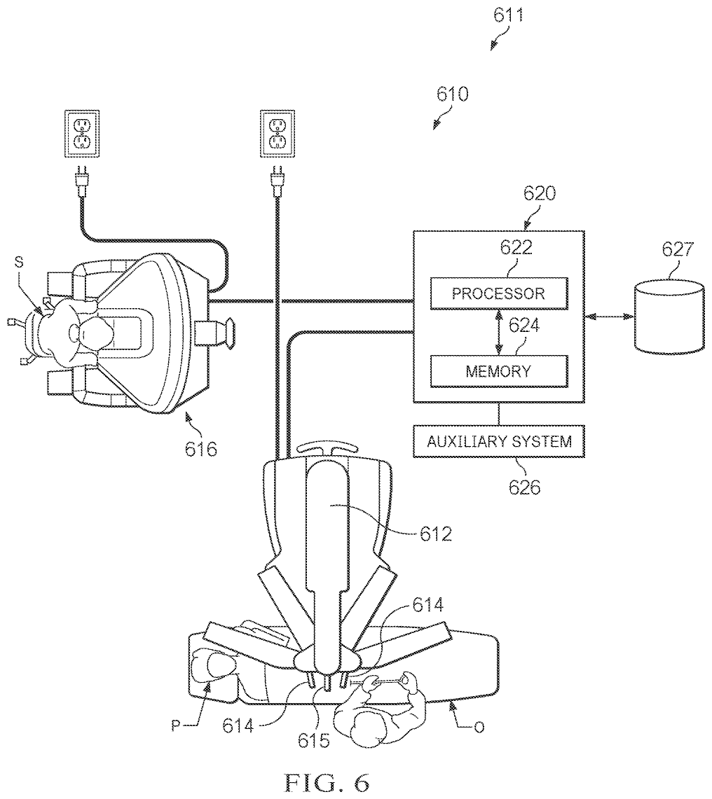

The medical procedure record 200 also includes data records A-G captured during the medical procedure. In some examples, as illustrated in FIG. 4 a medical procedure 300 may be documented using a variety of measurement and record systems 400 to generate data records 402 (e.g., the data records A-G) associated with the procedure 300 (e.g., a prior medical procedure). Measurement and record systems 400 may include, for example one or more sensor systems 410 associated with a robot-assisted medical system (e.g., medical system 610 of FIG. 6). In some examples, the sensor system 410 may include position, orientation, motion, and/or displacement sensor systems for manipulators or instruments coupled to manipulators of the robot-assisted medical system. In some examples, the sensor system 410 may include force sensor systems, clocks, motor encoders, energy usage sensors, user eye-tracking sensors, or other sensor systems that measure and/or record data about the manipulator or instruments. Measurement and record systems 400 may also or alternatively include one or more sensor systems 412 associated with monitored medical devices used in the medical procedure 300. In some examples, the sensor systems 412 may track position, orientation, motion, displacement, force, energy usage, duration of use, and/or other measures associated with the medical devices.

Measurement and record systems 400 may also or alternatively include one or more imaging systems 414 used during the medical procedure 300. In some examples, the imaging systems 414 may be in vivo imaging systems such as endoscopic imaging systems or ultrasound imaging systems used during the procedure 300. In some examples, the imaging systems 414 may be ex vivo imaging systems for the patient anatomy such as computed tomography (CT) imaging systems, magnetic resonance imaging (MRI) imaging systems, or functional near-infrared spectroscopy (fNIRS) imaging systems used during the procedure 300. In some examples, the imaging systems 414 may be environment imaging systems such as optical imaging systems that track the position and movement of manipulators, instruments, equipment, and/or personnel in the environment of the patient during the procedure 300.

Measurement and record systems 400 may also or alternatively include one or more audio systems 416 used during the medical procedure 300. The audio systems may capture and record audio from the personnel in the medical area of the procedure 300, the operator performing the procedure 300, the patient, and/or equipment in the medical area of the procedure 300. Measurement and record systems 400 may also or alternatively include one or more in-procedure patient monitoring systems 418 used during the medical procedure 300. The patient monitoring systems 418 may include, for example, respiration, cardiac, blood pressure, anesthesia, insufflation, and/or patient/table orientation monitoring systems. Measurement and record systems 400 may also or alternatively include one or more patient outcome record systems 420 that may be referenced after the procedure 300 is complete. Patient outcome record systems 420 may record information about post-procedure hospitalization duration, complications, positive outcomes, negative outcomes, mortality, or other post-procedure information about the patient. Measurement and record systems 400 may also or alternatively include one or more procedure skills record systems 422 that capture and record objective performance indicators for the clinician that performs the procedure 300.

The data records 402 may include the data generated by the measurement and record systems 400. For example, data records 430 may record the position, orientation, movement, and/or displacement of instruments (e.g., instruments 614) controlled by a robot assisted manipulator or by manual operation. In some examples, data records 432 may record the position, orientation, movement, and/or displacement of a robot-assisted manipulator assembly (e.g., 612) including any arms of the manipulator during the procedure 300. In some examples, data records 434 may record the position, orientation, movement, and/or displacement of an imaging system, such as an endoscopic or other in vivo or ex vivo imaging system, during the procedure 300. In some examples, data records 436 may record the position, orientation, movement, and/or displacement of an operator input device (e.g., 636) during the procedure 300. In some examples, data records 438 may record the position, orientation, movement, and/or displacement of an operator (e.g., surgeon S) directing the control of an instrument during the procedure 300. For example, the data records 438 may record motion of the operator's hands or track head disengagement from an operator console. In some examples, data records 440 may record the position, orientation, movement, and/or displacement of one or more members of a medical team involved with the procedure 300. In some examples, data records 442 may record aspects of the initial set-up of the procedure 300, including the position and arrangement of the robot-assisted manipulator assembly, patient port placement, and the location of peripheral equipment. In some examples, data records 444 may include records of the location, frequency, and amount of energy provided to or delivered by instruments (e.g. ablation instruments) during the procedure 300. In some examples, data records 446 may include records of instrument changes during the procedure 300. In some examples, data records 448 may include time-based records that capture dwell times, idle times, and/or duration or speed of an action during the procedure 300. In some examples, data records 450 may capture aspects of workflow including the quantity and/or sequence of actions during the procedure 300. For example, the data records 450 may include sequences of position, orientation, movements, and/or displacements associated with a discrete activity. In some examples, data records 452 may capture errors, difficulties, incidents, or other unplanned episodes, such as manipulator arm collisions, during the procedure 300, conditions leading to conversions during the procedure from a robot-assisted surgery to an open surgery, conditions leading to conversions during the procedure from a robot-assisted surgery to a laparoscopic surgery, or conditions leading to conversions during the procedure from a laparoscopic surgery to an open surgery. In some examples, data records 454 may capture aspects of the anatomic environment including size of organs, incisions, and/or treatment delivery areas. Other aspects of the anatomic environment that may be recorded include pelvic width, distance between anatomic structures, and/or locations of vasculature. In some examples, data records 456 may include interventional consequences such as measures of bleeding, smoke, tissue movement, and or tissue color change. In some examples, data records 458 may include a catalog of the key skills to perform the procedure 300, the relevant object performance indicators for experienced clinicians that perform the same type of procedure, and objective performance indicators of the clinician who performed the procedure 300.

With reference again to FIG. 3, in parameterized medical procedure record 200, the data records A-G (e.g., data records 430-456) have been associated with actions 244, 246, 248 performed during the medical procedure. The actions have been associated with parameters 250, 252, 254. 256 through, for example, the parameterization method described in U.S. Provisional Patent Application No. 63/320,538. In this example the actions and parameters have, optionally, been further grouped into segments 240, 242. For example, procedure segments may include sequences or groups of actions associated with ablation, stapling, suturing, dissection, tissue resection, anastomosis, camera control, instrument wrist control, setting changes, or tool changes that occur once or multiple times during a medical procedure. In this example, a procedure may include segment 240 and segment 242. The segment 240 may include actions 244 and 246. The action 244 may include two parameters 250 and parameter 252. The parameter 250 is or is determined from the single data record A. The parameter 252 is or is determined from three data records B-D. The action 246 may include a single parameter 254 that is or is determined from the data record E. The segment 242 includes a single action 248, and the action 248 includes a single parameter 256. The parameter 256 is or is determined from data record F and data record G.

As an example, the procedure record 200 may include segment 240 that is a tissue resection segment and segment 242 that is an ablation segment. The suturing segment 240 may include the actions of cutting tissue at action 244 and moving the cut tissue at action 246. The cutting action 244 may include a parameter 250 that includes a data record A that includes identification information for the cutting instrument. The cutting action 244 may include a parameter 252 that includes a data record B that includes the position and orientation of the end effector of the cutting instrument at the start of the cutting, a data record C that includes the position and orientation of the end effector of the cutting instrument at the conclusion of the cutting, and a data record D that includes a time duration between the start and conclusion of the cutting. The tissue moving action 246 may include the parameter 254 that includes a data record E that includes a distance the tissue is moved. The ablation segment 242 incudes a single action 248 of ablating tissue which includes the parameter 256 that is associated with a data record F for a power level and a data record G for a duration.

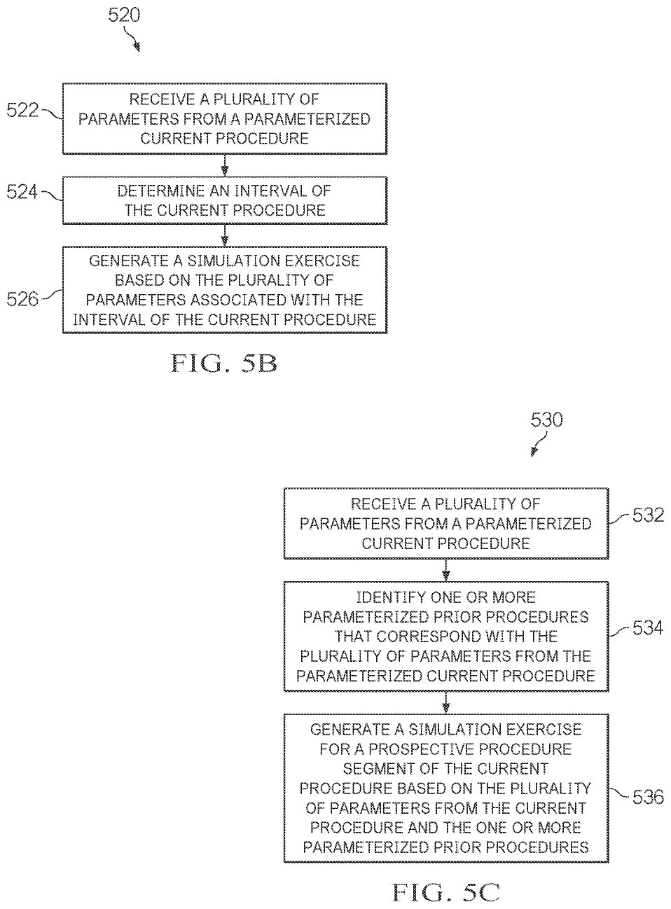

Referring again to FIG. 1, at the process 106, the simulation exercise may be determined based on the user profile and the parameterized prior procedure catalog, database or set. FIG. 5A provides an example of a method 500 for customizing a simulation procedure. The customized simulation procedure may, for example, allow a user to experience a difficult case previously performed by an expert. The experience may be customized based on the user's experience to include guidance or other assistance. The experience may also be customized, scaled, or otherwise adapted to the user's schedule of upcoming procedures. In some examples, the experience may be customized, based on the user's prior performance assessments, to include remediation exercises to correct performance or to include increasingly difficult exercises to expand the user's capabilities. The processes illustrated in FIG. 5A may be performed in a different order than the order shown in FIG. 5A, and one or more of the illustrated processes might not be performed in some embodiments of method 500. Additionally, one or more processes that are not expressly illustrated in FIG. 5A may be included before, after, in between, or as part of the illustrated processes. In some embodiments, one or more of the processes of method 500 may be implemented, at least in part, in the form of executable code stored on non-transitory, tangible, machine-readable media that when run by one or more processors (e.g., the processors of a control system) may cause the one or more processors to perform one or more of the processes. The process 106 of FIG. 1 may be manual in which case a clinician or other human operator(s) determines the simulation exercise; fully-automated in which case a control system determines the simulation exercise, or semi-automated in which case some parts of the process 106 are performed by a human operator and other parts are performed by a control system.

At a process 502, a user experience factor may be accessed or otherwise determined. A user experience factor may include a skill development subject for the user to improve a clinical skill or may include a type of future procedure scheduled for the user to allow the user to prepare for a future experience. Additionally or alternatively, the experience factor may include a factor associated with a past or upcoming procedure such as the sequencing of procedure workflow steps or anatomic parameters such as patient size or condition. The experience factor may be accessed or determined based on information in the user profile, information received from the user or other operator (e.g. a trainer), or any other source of information about skill development needs.

In an example in which the user experience factor includes one or more subjects or areas for skill development, the experience factor may be determined from user profile and/or the parameterized prior procedure information. For example, prior procedure information 152 from the user profile 150 may be compared with surgical objective performance indicators 458 from one or more prior procedures 300 to assess a user's skill level and identify needed skill development areas. For example, the user skill metrics may be compared to objective performance indicators or performance benchmarks to identify areas of weakness or opportunities to strengthen skills. For example, benchmarks or indicators for the skill of tissue ablation may include a measure of the amount of smoke generated, a measure of the amount of tissue color change, a measure of time to complete the procedure, and/or a measure of the number of clamping actions performed. Comparing the user's skill metrics for these benchmark skills may indicate that the user should complete simulation training to improve ablation skills. As another example, user skill metrics may be compared to the skills required for upcoming procedures types based on the user's schedule of upcoming or prospective procedures. Comparing the user's skill metrics for the skills required for upcoming procedure types may indicate that the user should complete simulation training to obtain or improve skills needed for those types of upcoming procedures. As another example, user skill metrics may be compared to the difficulty of upcoming procedures based on the user's schedule of upcoming procedures. Comparing the user's skill metrics to the difficulty of upcoming procedures may indicate that the user should complete simulation training to obtain or improve skills needed the level of upcoming difficulty. As another example, user patient history may be compared to the characteristics of the patients scheduled for upcoming procedures. Comparing the patient history to the characteristics of patients scheduled for upcoming procedure types may indicate that the user should complete simulation training to obtain or improve skills needed to match the size, gender, pathology type, complications, or other characteristics of the scheduled patients. As another example, the user's team composition history may be compared to the teams scheduled for the user's upcoming procedures. Comparing the user's team composition history to the team composition, skill level, and experience level for upcoming procedures may indicate that the user should complete simulation training to obtain or improve skills needed for effectively work with the planned teams. As another example, the user's skill metrics may be evaluated to determine the next level skills for incremental skill growth. As another example, the user may self-identify areas for needed growth or improvement based on the user's profile or based on the parameterized prior procedures. As another example, a trainer, key operating leader, expert or other mentor figure may identify areas for the user's growth or improvement based on the user's profile or based on the parameterized prior procedures.

At a process 504, one or more parameterized prior procedures may be identified from the set of parameterized prior procedures to address the identified skill development needs. For example, if an upcoming procedure involves a patient with underlying conditions not previously experienced by the user, parameterized prior procedures involving real or simulated patients with the same underlying conditions may be identified. As another example, if an upcoming procedure involves use of a new robot-assisted medical system that is different from the systems previously used by the user, parameterized prior procedures using the new system may be identified. In some examples, the parameterized prior procedures may be the procedures of experts, key operational leaders, trainers, peers, or even the user that are associated with efficient, minimal error, or otherwise successful prior procedures. In some examples, the parameterized prior procedures may be procedures of the user or others that included errors, suboptimal performance, inefficiencies, or other issues that may be studied or retired to improve the outcome.

At an optional process 506, aspects of the identified parameterized prior procedure may be modified. For example, segments, actions, or parameters from the identified prior procedure may be omitted or combined with segments, actions, or parameters from other identified prior procedures. In some examples, the identified parameterized prior procedure may be scaled based patient dimensions, gender, age, or other conditions that correspond to the user's skill development needs. In some examples, the identified parameterized prior procedure may be modified to allow for additional team members to participate in the simulation. In some examples, the identified parameterized prior procedure may be modified to include synthetic team members. In some examples, the port placements used for a parameterized prior robot-assisted medical procedure may be modified. In some examples, the identified parameterized prior procedure may be modified to include different systems, devices, or instruments.

At a process 508 the simulation exercise may be generated. The simulation exercise may allow the user to virtually experience the parameterized prior procedure, including a simulated user interface that provides visual, audio, and/or haptic simulation of the identified prior procedure. The simulation exercise may include some or all the parameters from the parameterized prior procedure, based on the data records obtained during the prior procedure. The simulation exercise may simulate the robot-assisted medical systems and instruments, laparoscopic instruments, and/or open procedure instruments used in the identified prior procedure. Image data, audio data, force transmission data, patient monitoring data, and/or patient outcome data recorded or gathered and parameterized from the identified prior procedure may be included in the simulation exercise. In some examples, image data, audio data, force transmission data, patient monitoring data, and/or patient outcome data may be artificially generated and included in the simulation exercise to create a synthetic or hybrid synthetic-recorded environment and experience. The simulation exercise may be interactive and responsive to user inputs. In some examples, the simulation exercise may be presented to the user at a simulated user console that includes user interface components of an operator input system (e.g., operator input system 616) including display systems, audio systems, and user input control devices. In some examples, the simulation exercise may be presented to the user at an actual user console (e.g. operator input system 616) that is operating in a simulation mode. In some examples, the simulation exercise may be adapted for presentation to the user on a laptop, tablet, phone or other user input device that may include a display, user input control devices, a control system, a memory, and/or other components that support the visual, audio, and/or haptic user experience. In some examples, a simulation may dynamically adapted based on user preference. For example, after a simulation is started with a first instrument as used by a first mentor clinician in a first prior procedure, the user may elect to change the simulation to use a second instrument as used by a second mentor clinician in a second prior procedure. In some examples, the simulation may include an inanimate anatomic model or a synthetic tissue model customized and built for the simulation. For example, a synthetic model may include a custom anatomical defect or customized instrument port placements relative to a defect.

At an optional process 510, model operator inputs for performing the simulation exercise may be determined from the parameterized prior procedure. The model operator inputs may include hand, arm, body, eye, head, foot or other motions or behaviors that are used to generate or are otherwise associated with the data records on which the parameters of the simulation exercise are based. For example, the parameters associated with the action of suturing tissue in the prior procedure were determined from the data records or procedural information records associated with a series of steps in performing the act of suturing. Thus, the model operator inputs associated with suturing based on the parameters of the prior procedure may include selecting an instrument with the same identity to be used for grasping the suturing filament, selecting the same manipulator arm used to control the instrument, applying a same or similar force to grasp the filament, rotating the wrist joint of the instrument a same or similar amount, the completing the rotation in the same or similar duration of time, releasing the filament at a same or similar position and orientation of the instrument. The model operator inputs may generate data records that are the same as or within a predetermined range of the corresponding data records for the prior procedure. In some examples, the user's inputs in the simulation may be evaluated against or compared to the model operator inputs to provide guidance, error reports, success confirmation reports, or other indicia that the user's input is the same as, similar to, or different from the model operator inputs.

At an optional process 512, guidance for performing the model inputs may be generated. For example, guidance may include simulated graphics for visual display to the user during the simulation exercise to explain or demonstrate the model inputs. The simulated graphics may include ghost tool illustrations. Guidance may also include haptic forces delivered to operator input devices or audio guidance. Guidance may include hand-over-hand demonstrations that allow the user to follow the guided hand motions. Guidance may include pop-up graphical, textual information, and/or highlighting or emphasized graphics. Guidance may include graphical indicator, picture-in-picture displays, and/or synthetic or pre-recorded videos of alternative techniques. Guidance may include the ability to rewind or fast forward the guidance. The guidance may be responsive to measured or sensed user inputs or team member inputs.

In some examples, the methods described herein may be used to generate customized camera control simulations. From parameterized prior procedures, a sequence of camera targets (e.g., locations in the field of view on which the camera is focused or orientations of the camera) may be determined, and a simulation may be generated that prompts the user to take actions that follow the same sequence of camera movements. In some examples, the methods described herein may be used to generate customized suturing simulations. From parameterized prior procedures, a sequence of needle positions, orientations, and movements may be determined, and a simulation may be generated that leads the user to take actions that follow the same or a similar sequence of positions, orientations, and movements. In some examples, the methods described herein may be used to generate customized dissection simulations. From parameterized prior procedures, a sequence of instrument positions, orientations, and motions may be determined, and a simulation may be generated that leads the user to take actions that follow the same or a similar sequence of positions, orientations, and movements. In some examples, the methods described herein may be used to generate customized energy delivery. From parameterized prior procedures, locations and amounts of ablation energy may be determined, and a simulation may be generated that teaches the user to deliver energy with the same parameters. In some examples, the methods described herein may be used to perform a set-up procedure including the port-placement for robot-assisted instruments. From parametrized prior procedures, initial manipulator assembly set-up or arm arrangement and/or optimized locations for port placements may be determined, and a simulation may be generated that leads the user to select the same or similar manipulator assembly set-up or port placement locations.

In another example of the process 106, the flowchart of FIG. 5B illustrates a method 520 for customizing a simulation exercise. In this example, the customized simulation exercise may be based on a current medical procedure (e.g., diagnostic, therapeutic, or surgical procedure being conducted real-time on a patient). In some examples, the simulation experience may be experienced contemporaneously with the current medical procedure or may be experienced after the completion of the current medical procedure. The simulation experience may, for example, allow a clinician performing the current procedure to virtually re-experience or repeat a segment of the current procedure or may allow a surgical trainee to virtually experience the segment contemporaneously with the current procedure.

As the current medical procedure is being performed, a parameterized medical procedure record (e.g., the record 200) may be generated in real-time for a current procedure. At a process 522, a plurality of parameters may be received for the parameterized current procedure. For example, parameters from the medical record of the current procedure may include procedure information (e.g. procedure information 202) or parameters associated with the segments and actions of the procedure.

At a process 524, an interval of the current procedure may be determined. The interval may be determined or identified in any of various ways. For example, the interval may have a predetermined fixed duration relative to a duration trigger (e.g., the two minutes of the current procedure prior to engaging a triggering switch on a user interface or control device). In other examples, the interval of the current procedure may have a user defined duration (e.g., the prior ninety seconds before a trigger). In other examples, the interval of the current procedure may be marked by a user engaging a trigger switch at a start time of the interval and the end time of the interval. In other examples, the interval of the current procedure may be marked by rewinding a video recording of the current procedure from and an interval end frame to an interval start frame. In other examples, the interval may be determined by image analysis identifying a triggering action of a tool or tissue in an endoscopic image associated with a start or stop action of the interval. In other examples, the interval may be determined by kinematic recognition of a triggering event such as a motion or command to move a robotic arm or tool attached to a robotic arm.

At a process 526, a simulation exercise may be generated based on the plurality of parameters associated with the interval of the current procedure. The simulation exercise may be an augmented reality or virtual reality simulation that allows a user to experience the interval of the current procedure in a simulated environment. The simulation exercise may be provided, for example, to a trainee who has viewed the current procedure on a secondary operator's console and would like to now perform the witnessed procedure segment in a simulated environment. The simulation exercise may be performed by the trainee while the current procedure is continued by the original clinician or may be performed at a later time (including repeatedly), after the current procedure is concluded. In some examples, the simulation exercise may be provided to the original clinician who performed the current procedure, after the conclusion of the current procedure, to allow the clinician to practice the identified portion of the procedure one or more times following the current procedure. As an example, if the current procedure includes a dissection segment, the interval of the current procedure that includes the dissection section may be identified. The generated simulation would allow a trainee or the original clinician to virtually experience the identified interval that includes the dissection section, under the same parameters as the original procedure. The virtual experience may include guidance (e.g., haptic guidance, visual guidance) that encourages the user to replicate the same hand motions and tool manipulations as in the original procedure. As another example, if the current procedure includes a camera adjustment, the interval of the current procedure that includes the camera adjustment may be identified. The generated simulation would allow a trainee or the original clinician to virtually experience the identified interval that includes the camera adjustment, under the same parameters as the original procedure. The virtual experience may include guidance (e.g., haptic guidance, visual guidance) that encourages the user to replicate the same hand motions and camera manipulations as in the original procedure.

In another example of the process 106, the flowchart of FIG. 5C illustrates a method 530 for customizing a simulation exercise. In this example, the customized simulation exercise may be based on a current medical procedure (e.g., diagnostic, therapeutic, or surgical procedure being conducted real-time on a patient) but allow a user to preview a prospective or future segment of the current procedure. The simulation experience may be experienced contemporaneously with the current medical procedure, such as during a pause in the current medical procedure. The simulation experience may, for example, allow a clinician performing the current procedure to virtually experience a predictable segment of the current procedure as determined and modeled from prior procedures of the same type by the same or a different clinician.

As the current medical procedure is being performed, a parameterized medical procedure record (e.g., the record 200) may be generated in real-time for a current procedure. At a process 532, a plurality of parameters may be received for the parameterized current procedure. For example, parameters from the medical record of the current procedure may include procedure information (e.g. procedure information 202) or parameters associated with the segments and actions of the procedure.

At a process 534, one or more prior procedures that correspond with the plurality of parameters from the parameterized current procedure may be identified. The identified prior procedure may have the same or similar parameters including, for example, the same tools being used, the same robotic manipulator set-up configuration, the same instrument set up, and similar patient characteristics (e.g., BMI, stage of disease progression).

At a process 536, a simulation exercise may be generated for a prospective segment of the current procedure based on the plurality of parameters from the current procedure and the one or more parameterized prior procedures. The simulation exercise may be an augmented reality or virtual reality simulation that allows a clinician performing the current procedure to experience a prospective segment of the current procedure in a simulated environment. The simulation exercise may include guidance (e.g., haptic guidance, visual guidance) that encourages the user to practice or replicate the same hand motions and tool manipulations as in the prior procedure which had similar parameters. In some examples, the simulation exercise may allow a clinician to preview a segment that is prone to instrument or arm collisions and train with the prior procedure parameters to learn to avoid the collisions. In some examples, the simulation exercise may allow a clinician to practice camera movements in anticipation of an upcoming procedure segment that is preferably performed under a different camera angle. In some examples, the simulation exercise may allow a clinician to practice tool wrist motions in anticipation of an upcoming segment of the current procedure. The simulation exercise may occur during a pause in the current procedure, using the same user control devices that are removed from an instrument following mode so that motion of the control devices does not active the surgical tools within the patient. The simulation exercise may allow a user to perform the segment simulation close in time to the subsequent performance of the actual procedure segment using the same tools, same set-up configuration, and same patient characteristics.

The medical procedures and simulations described herein may be performed with a variety of manual or robot-assisted technologies. FIGS. 6-8 together provide an overview of a robot-assisted medical system 610 that may be used in, for example, medical procedures or simulations including diagnostic, therapeutic, or surgical procedures. The medical system 610 is located in a medical environment 611. The medical environment 611 is depicted as an operating room in FIG. 6. In other embodiments, the medical environment 611 may be an emergency room, a medical training environment, a medical laboratory, or some other type of environment in which any number of medical procedures or medical training procedures may take place. In still other embodiments, the medical environment 611 may include an operating room and a control area located outside of the operating room.

In one or more embodiments, the medical system 610 may be a teleoperational medical system that is under the teleoperational control of a surgeon. In alternative embodiments, the medical system 610 may be under the partial control of a computer programmed to perform the medical procedure or sub-procedure. In still other alternative embodiments, the medical system 610 may be a fully automated medical system that is under the full control of a computer programmed to perform the medical procedure or sub-procedure with the medical system 610. One example of the medical system 610 that may be used to implement the systems and techniques described in this disclosure is the da Vinci® Surgical System manufactured by Intuitive Surgical Operations, Inc. of Sunnyvale, California.

As shown in FIG. 6, the medical system 610 generally includes an assembly 612, which may be mounted to or positioned near an operating table O on which a patient P is positioned. The assembly 612 may be referred to as a patient side cart, a surgical cart, or a surgical robot. In one or more embodiments, the assembly 612 may be a teleoperational assembly. The teleoperational assembly may be referred to as, for example, a manipulating system and/or a teleoperational arm cart. A medical instrument system 614 and an endoscopic imaging system 615 are operably coupled to the assembly 612. An operator input system 616 allows a surgeon S or other type of clinician to view images of or representing the surgical site and to control the operation of the medical instrument system 614 and/or the endoscopic imaging system 615.

The medical instrument system 614 may comprise one or more medical instruments. In embodiments in which the medical instrument system 614 comprises a plurality of medical instruments, the plurality of medical instruments may include multiple of the same medical instrument and/or multiple different medical instruments. Similarly, the endoscopic imaging system 615 may comprise one or more endoscopes. In the case of a plurality of endoscopes. the plurality of endoscopes may include multiple of the same endoscope and/or multiple different endoscopes.

The operator input system 616 may be located at a surgeon's control console, which may be located in the same room as operating table O. In one or more embodiments, the operator input system 616 may be referred to as a user control system. In some embodiments, the surgeon S and the operator input system 616 may be located in a different room or a completely different building from the patient P. The operator input system 616 generally includes one or more control device(s), which may be referred to as input control devices, for controlling the medical instrument system 614 or the imaging system 615. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same Cartesian degrees of freedom as the medical instrument(s) of the medical instrument system 614 to provide the surgeon S with telepresence, which is the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon S with telepresence. In some embodiments, the control device(s) are manual input devices that move with six degrees of freedom, and which may also include an actuatable handle for actuating instruments (for example, for closing grasping jaw end effectors, applying an electrical potential to an electrode, delivering a medicinal treatment, and actuating other types of instruments). Therefore, the degrees of freedom and actuation capabilities of the control device(s) are mapped to the degrees of freedom and range of motion available to the medical instrument(s).

The assembly 612 supports and manipulates the medical instrument system 614 while the surgeon S views the surgical site through the operator input system 616. An image of the surgical site may be obtained by the endoscopic imaging system 615, which may be manipulated by the assembly 612. The assembly 612 may comprise multiple endoscopic imaging systems 615 and may similarly comprise multiple medical instrument systems 614 as well. The number of medical instrument systems 614 used at one time will generally depend on the diagnostic or surgical procedure to be performed and on space constraints within the operating room, among other factors. The assembly 612 may include a kinematic structure of one or more non-servo controlled links (e.g., one or more links that may be manually positioned and locked in place, generally referred to as a manipulator support structure) and a manipulator. When the manipulator takes the form of a teleoperational manipulator, the assembly 612 is a teleoperational assembly. The assembly 612 includes a plurality of motors that drive inputs on the medical instrument system 614. In an embodiment, these motors move in response to commands from a control system (e.g., control system 620). The motors include drive systems which when coupled to the medical instrument system 614 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 614 may include end effectors having a single working member such as a scalpel, a blunt blade, an optical fiber, or an electrode. Other end effectors may include, for example, forceps, graspers, scissors, or clip appliers.

The medical system 610 also includes a control system 620. The control system 620 includes at least one memory 624 and at least one processor 622 (which may be part of a processing unit) for effecting control between the medical instrument system 614, the operator input system 616, and other auxiliary systems 626 which may include, for example, imaging systems, audio systems, fluid delivery systems, display systems, illumination systems, steering control systems, irrigation systems, and/or suction systems. A clinician may circulate within the medical environment 611 and may access, for example, the assembly 612 during a set up procedure or view a display of the auxiliary system 626 from the patient bedside. In some embodiments, the auxiliary system 626 may include a display screen that is separate from the operator input system 616. In some examples, the display screen may be a standalone screen that is capable of being moved around the medical environment 611. The display screen may be orientated such that the surgeon S and one or more other clinicians or assistants may simultaneously view the display screen.

Though depicted as being external to the assembly 612 in FIG. 6, the control system 620 may, in some embodiments, be contained wholly within the assembly 612. The control system 620 also includes programmed instructions (e.g., stored on a non-transitory, computer-readable medium) to implement some or all the methods described in accordance with aspects disclosed herein. While the control system 620 is shown as a single block in the simplified schematic of FIG. 6, the control system 620 may include two or more data processing circuits with one portion of the processing optionally being performed on or adjacent the assembly 612, another portion of the processing being performed at the operator input system 616, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 620 supports wireless communication protocols such as Bluetooth, IrDA, HomeRF, IEEE 802.11, DECT, and Wireless Telemetry.

The control system 620 is in communication with a database 627 which may store one or more medical procedure records. The database 627 may be stored in the memory 624 and may be dynamically updated. Additionally or alternatively, the database 627 may be stored on a device such as a server or a portable storage device that is accessible by the control system 620 via an internal network (e.g., a secured network of a medical facility or a teleoperational system provider) or an external network (e.g., the Internet). The database 627 may be distributed throughout two or more locations. For example, the database 627 may be present on multiple devices which may include the devices of different entities and/or a cloud server. Additionally or alternatively, the database 627 may be stored on a portable user-assigned device such as a computer, a mobile device, a smart phone, a laptop, an electronic badge, a tablet, a pager, and other similar user devices.

In some embodiments, the control system 620 may include one or more servo controllers that receive force and/or torque feedback from the medical instrument system 614. Responsive to the feedback, the servo controllers transmit signals to the operator input system 616. The servo controller(s) may also transmit signals instructing assembly 612 to move the medical instrument system(s) 14 and/or endoscopic imaging system 615 which extend into an internal surgical site within the patient body via openings in the body. Any suitable conventional or specialized servo controller may be used. A servo controller may be separate from, or integrated with, assembly 612. In some embodiments, the servo controller and assembly 612 are provided as part of a teleoperational arm cart positioned adjacent to the patient's body.

The control system 620 can be coupled with the endoscopic imaging system 615 and can include a processor to process captured images for subsequent display, such as to a surgeon on the surgeon's control console, or on another suitable display located locally and/or remotely. For example, where a stereoscopic endoscope is used, the control system 620 can process the captured images to present the surgeon with coordinated stereo images of the surgical site as a field of view image. Such coordination can include alignment between the opposing images and can include adjusting the stereo working distance of the stereoscopic endoscope.

In alternative embodiments, the medical system 610 may include more than one assembly 612 and/or more than one operator input system 616. The exact number of assemblies 612 will depend on the surgical procedure and the space constraints within the operating room, among other factors. The operator input systems 616 may be collocated or they may be positioned in separate locations. Multiple operator input systems 616 allow more than one operator to control one or more assemblies 612 in various combinations. The medical system 610 may also be used to train and rehearse medical procedures.

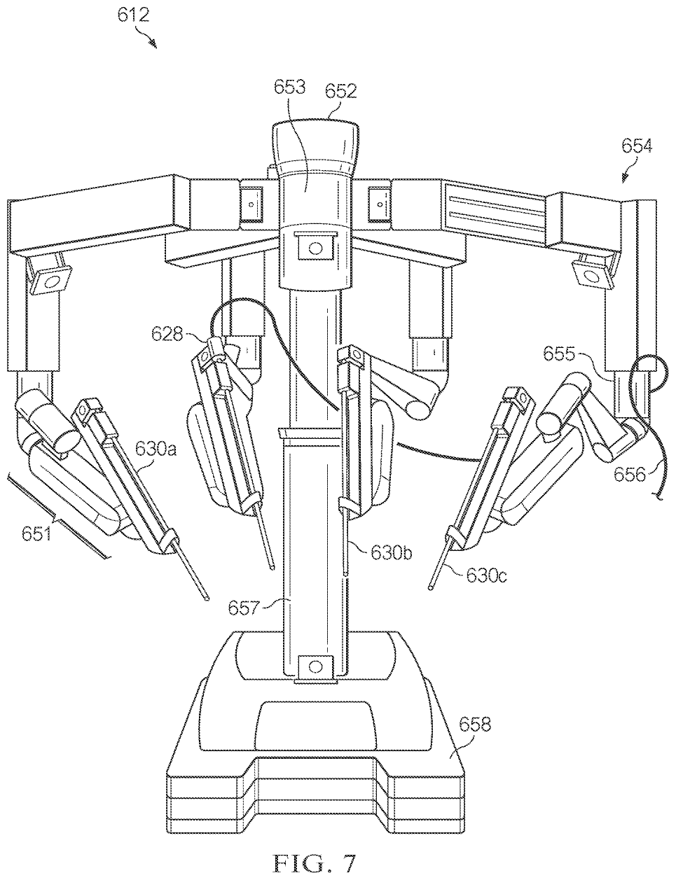

FIG. 7 is a perspective view of one embodiment of a manipulator assembly 612 which may be referred to as a manipulating system, patient side cart, surgical cart, teleoperational arm cart, or surgical robot. The assembly 612 shown provides for the manipulation of three surgical tools 630a, 630b, and 630c (e.g., medical instrument systems 614) and an imaging device 628 (e.g., endoscopic imaging system 615), such as a stereoscopic endoscope used for the capture of images of the site of the procedure. The imaging device 628 may transmit signals over a cable 656 to the control system 620. Manipulation is provided by teleoperative mechanisms having a number of joints. The imaging device 628 and the surgical tools 630a-c can be positioned and manipulated through incisions in the patient so that a kinematic remote center is maintained at the incision to minimize the size of the incision. Images of the surgical site can include images of the distal ends of the surgical tools 630a-c when they are positioned within the field-of-view of the imaging device 628. The imaging device 628 and the surgical tools 630a-c may each be therapeutic, diagnostic, or imaging instruments.

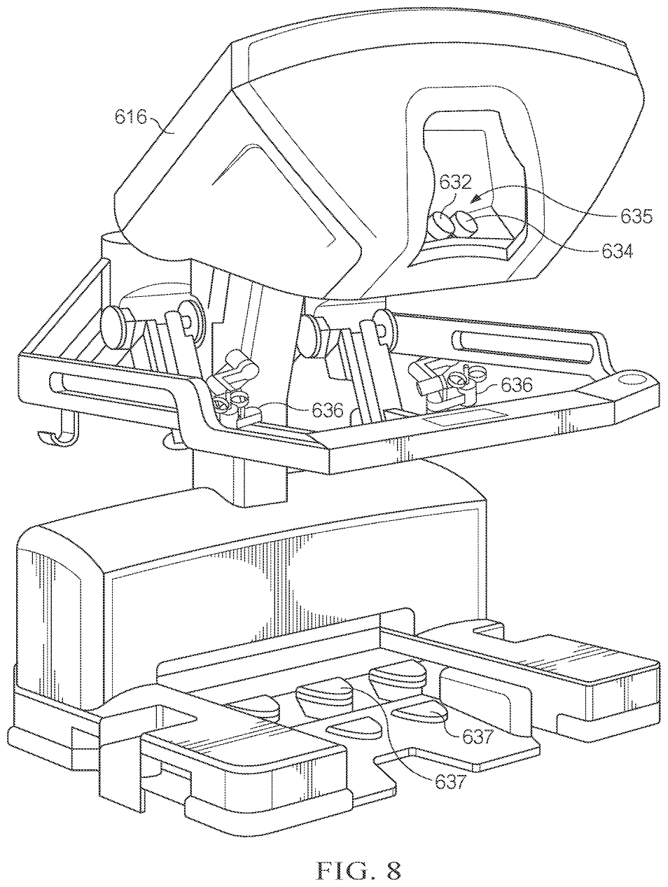

FIG. 8 is a perspective view of an embodiment of the operator input system 616 at the surgeon's control console. In some examples, the operator input system 616 may be a simulation medical system, decoupled from control of the medical instrument or manipulation assembly. The simulation medical system may be coupled to the control system to access the catalog of parameterized prior medical procedures, user profiles, or other information used to generate and deliver the simulation exercise.

In this example, the operator input system 16 includes a left eye display 632 and a right eye display 634 for presenting the surgeon S with a coordinated stereo view of the surgical environment that enables depth perception. The left and right eye displays 632. 634 may be components of a display system 635. In other embodiments, the display system 635 may include one or more other types of displays. In some embodiments, image(s) displayed on the display system 635 may be separately or concurrently displayed on at least one display screen of the auxiliary system 626.

The operator input system 616 further includes one or more input control devices 636, which in turn cause the assembly 612 to manipulate one or more instruments of the endoscopic imaging system 615 and/or the medical instrument system 614. The input control devices 636 can provide the same Cartesian degrees of freedom as their associated instruments to provide the surgeon S with telepresence, or the perception that the input control devices 636 are integral with said instruments so that the surgeon has a strong sense of directly controlling the instruments. Therefore, the degrees of freedom of each input control device 636 are mapped to the degrees of freedom of each input control device's 636 associated instruments (e.g., one or more of the instruments of the endoscopic imaging system 615 and/or the medical instrument system 614.). To this end, position, force, and tactile feedback sensors (not shown) may be employed to transmit position, force, and tactile sensations from the medical instruments, e.g., the surgical tools 630a-c or the imaging device 628, back to the surgeon's hands through the input control devices 636. Additionally, the arrangement of the medical instruments may be mapped to the arrangement of the surgeon's hands and the view from the surgeon's eyes so that the surgeon has a strong sense of directly controlling the instruments. Input control devices 637 are foot pedals that receive input from a user's foot. Aspects of the operator input system 616, the assembly 612, and the auxiliary systems 626 may be adjustable and customizable to meet the physical needs, skill level, or preferences of the surgeon S.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions. Not all the illustrated processes may be performed in all embodiments of the disclosed methods. Additionally, one or more processes that are not expressly illustrated in may be included before, after, in between, or as part of the illustrated processes. In some embodiments, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for imaging, any of a variety of anatomic systems, including the lung, colon, the intestines, the stomach, the liver, the kidneys and kidney calices, the brain, the heart, the circulatory system including vasculature, and/or the like. While some embodiments are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in embodiments of this disclosure may be implemented in software to execute on a processor of a computer system such as control processing system. When implemented in software, the elements of the embodiments of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet. Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A medical system comprising:
a display system;
an operator input device; and
a control system in communication with the display system and the operator input device, wherein the control system comprises:
a processor; and
a memory comprising machine readable instructions that, when executed by the processor, cause the control system to:
access an experience factor for a user;
reference a set of parameterized prior procedures;
identify a parameterized prior procedure associated with the experience factor from the set of parameterized prior procedures;
generate a simulation exercise that includes a plurality of parameters from the parameterized prior procedure; and
determine model inputs to the operator input device that are associated with the plurality of parameters.

2. The medical system of claim 1, wherein the experience factor includes a skill development subject.

3. The medical system of claim 1, wherein the experience factor includes a type of future procedure scheduled for the user.

4. The medical system of claim 1, wherein the experience factor is determined from a prior procedure performed by the user.

5. The medical system of claim 1, wherein the experience factor includes an anatomic parameter.

6. The medical system of claim 1, wherein the experience factor is determined from a user profile.

7. The medical system of claim 1, wherein the machine readable instructions, when executed by the processor, further cause the control system to compare received input at the operator input device to the model inputs.

8. The medical system of claim 1, wherein the machine readable instructions, when executed by the processor, further cause the control system to scale the parameterized prior procedure.

9. The medical system of claim 1, wherein the machine readable instructions, when executed by the processor, further cause the control system to generate guidance for performing the model inputs.

10. The medical system of claim 1, wherein accessing the experience factor includes comparing a user skill profile to a schedule of prospective procedures.

11. The medical system of claim 1, wherein accessing the experience factor includes evaluating a user skill assessment against an objective performance indicator.

12. The medical system of claim 1, wherein the set of parameterized prior procedures includes parameterized prior procedures performed by an operator of the operator input device.

13. The medical system of claim 1, wherein the set of parameterized prior procedures includes parameterized prior procedures performed by a trainer.

14. The medical system of claim 1, wherein the plurality of parameters is determined from at least one of robotic system records, sensor data, in vivo imaging data, ex vivo imaging data, patient monitoring data, or patient outcome records.

15. The medical system of claim 1, wherein the plurality of parameters includes a position, orientation, or movement of a medical instrument or an imaging instrument.

16. (canceled)

17. The medical system of claim 1, wherein the plurality of parameters includes a position, orientation, or movement of an operator input device.

18. (canceled)

19. The medical system of claim 1, wherein the plurality of parameters includes a position, orientation, or movement of a team member.

20. The medical system of claim 1, wherein the plurality of parameters includes an initial manipulator assembly set-up arrangement.

21. The medical system of claim 1, wherein the plurality of parameters includes initial anatomic port placements.

22. The medical system of claim 1, wherein the plurality of parameters includes a location, frequency, or amount of delivered energy.

23-46. (canceled)
